(19) United States
(12) Patent Application Publication
(10) Pub. No.: US 2020/0282086 A1
(43) Pub. Date: Sep. 10, 2020

(54) SYSTEM AND METHOD FOR STERILIZATION OF FLUIDS

(71) Applicant: Lawrence Silverman, Newtown Square, PA (US)

(72) Inventor: Lawrence Silverman, Newtown Square, PA (US)

(21) Appl. No.: 16/597,268

(22) Filed: Oct. 9, 2019

Related U.S. Application Data
(60) Provisional application No. 62/716,591, filed on Aug. 9, 2018.

(51) Int. Cl. A61L 2/00; A61L 2/24; A61L 9/20; A61M 1/36; A61N 5/06

(52) U.S. Cl. CPC A61L 2/0047; A61N 2005/063; A61L 9/20; A61M 1/3681; A61N 5/0624; A61L 2202/11; A61L 2202/14; A61L 2202/21; A61L 2202/22; A61L 2209/12; A61L 2209/16; A61L 2209/111; A61M 2202/0423; A61N 2005/0661; A61L 2/24

(57) ABSTRACT

The invention relates to a system and method to sanitize a fluid while it is moving through a distribution system. The invention provides significant improvements in efficacy and reductions in cost over previous approaches. At least one embodiment of the invention is applicable to commercial, industrial, military, government, public and private facilities that employ forced-air ventilation systems, especially those with comparatively dense human occupancy. The invention has particular utility in hospitals, health care facilities, manufacturing facilities, offices, apartments and dormitories, military housing, afford and schools, as well as on ships, commercial airliners and in public and private transportation. At least one embodiment of the invention may be used to deliver UV-C to a specific surgical site to sanitize the site to prevent future infection.

### FIELD OF THE INVENTION

This invention relates to equipment used for disinfection. More specifically, this invention relates to the use of light in the UV-C spectrum as a disinfecting agent applied to moving fluids as well as to areas of an animate body, such as a person.

### BACKGROUND

Short-wave ultraviolet energy (“Tithonic” or “UV-C” radiation) has the ability to destroy a wide range of micro-organisms (such as for example e.g. viruses, bacteria, germs and other pathogens). These types of micro-organisms are common sources of infection, are extremely contagious and produce large consequent economic losses. Sufficient exposure to UV-disrupts the DNA of a micro-organism so that it is unable to reproduce. UV-C radiation is extremely effective at killing micro-organisms, whether in the air, suspended in a fluid, deposited on the surface of an object, or inside of an animate object.

UV-C has been used to disinfect water, air, and in specialized medical and industrial applications for many years. However UV-C technology has not been implemented in a standardized way that can be applied systematically on a building-wide basis in the forced air systems used for general HVAC distribution. The need is particularly acute in retrofit installations for healthcare, offices and public buildings. The invention is readily configured to provide a radiation field appropriate for virtually any size and system of forced-air ventilation and volume of air entering a space. The invention also includes methods to use UV-C to disinfect surfaces. This also and to incorporate UV-C to deliver disinfecting radiation to a site in an animate body, such as a surgical site or infection of a human body.

UV-C radiation can remove microorganism that include, but are not limited to:
Mold and Spores
Surgical-site infection (SSI)
Fungus
Viruses
Drug-resistant “super-bug” bacteria, such as MRSA (methicillin-resistant Staphylococcus aureus),
Airborne pathogens, such as tuberculosis and influenza (flu)
Cold germs
Allergens
Protozoa

At least one embodiment of the invention UV-C can be employed to eliminate pathogens in blood and other fluids used in medicine (including dentistry), biological (including greenhouses) and laboratory procedures.

### A. Mechanism of Anti-Microbial Operation

The energy of a UV photon varies inversely with the wavelength of the light; the shorter the wavelength, the more energetic the photon. The UV portion of the electromagnetic spectrum is classified according to wavelength as:
UV-A—400 nm-320 nm
UV-B—320 nm-290 nm
UV-C—290 nm-100 nm (includes “far UV-C” at <200 nm).

UV disinfection is preferably accomplished using UV-C because the high-energy photons in UV-C are more effective at breaking molecular bonds as well as rearranging electron configurations than UV-A or UV-B. UV-C functions as a disinfectant by using high-energy UV-C photons to break the molecular bonds in DNA material of a microorganism, rendering the organism inert or unable to reproduce. The longer the substance is exposed to UV-C light (“dwell time”), the greater the probability that all molecular bonds will have been destroyed by high energy UV-C photons.

The disinfection guidelines of the US Center for Disease Control and Prevention (“CDC”) list UV radiation between 200 and 328 nm as effective for disinfection, with a maximum of anti-bactericidal activity occurring between 240-280 nm (UV-C range). At these wavelengths, DNA damage (Thymine dimers) is induced at a high rate in the micoorganisms, and the cells become unable to reproduce.

UV-C exposure is a function of the output of the UV-C sources, the speed at which the fluid to be treated flows past them (i.e. the exposure time), and the square of the distance the fluid itself is separated from the UV-C sources. The amount of UV-C exposure is generally expressed in joules (one UV-C watt of energy for one second) per square meter at the point of measurement; or the equivalent: μ j/cm² (micro-joules per square centimeter). The amount of UV-C exposure needed to destroy a given microorganism is also a function of the size and structure of the organism itself.

Spores such as anthrax have a “cell wall” (like bacteria) as well as an outer “shell” that must be penetrated by the UV-C energy. Viruses such as influenza, the common cold, SARS, measles and small pox do not have a cell wall and are about five times more susceptible to UV-C radiation than spores. Bacteria with a cell wall such as tuberculosis, even extended drug resistant (XDR) TB, may be ten times more vulnerable to UV-C radiation than anthrax spores.

UV-C lamps required for microbial sterilization pose a potential health risk to human beings. Repeated or sustained exposure to UV-C radiation can damage the eyes and skin. Keratoconjunctivitis (external inflammation of the eye) and erythmea (reddening of the skin) can result from overexposure to UV-UV-C light is also known to induce skin cancer and cataracts.

Thus UV-C lamps must be installed in areas where humans are not directly exposed to UV-C light. The National Institutes for Occupational Safety and Health (NIOSH) recommends an upper limit on the amount of UV-C radiation for the safety of personnel in a room to be approximately 6 μj/cm² (i.e. 6 micro-joules per square centimeter) over a continuous eight-hour period. Although these standards may be modified from time to time, NIOSH guidelines must be considered in the design of all equipment and installations for human-occupied spaces.

### B. Previous Approaches to In-Room Air Sanitization

Any discussion of the prior or related art throughout the specification should in no way be considered as an admission that such prior art is widely known or forms part of common general knowledge in the field.

Microbial reduction occurs in air that passes directly through the UV-C field of a required strength. As an example, early efforts to use UV-C inside of rooms focused on installing UV-C lamps and light sources in wall and ceiling pendant light fixtures that would create a strong UV-C field across a room at a height above the “eye level” of people in the room (generally considered to be approximately 60-66 inches above the finished floor). However, in recent years, energy cost considerations have generally reduced ceiling heights, typically to eight feet, which makes it more difficult to achieve an effective UV-C dose in the upper part of the room (above 5 to 5.5 feet) without exceeding acceptable limits in the lower part of the room (at average human eye level and below). The UV-C beam is focused in a horizontal plane.

Additionally, the flow of air in a given room or area is not readily controlled. Air flow is subject to convection currents and the effects of ventilation, emanating from both inside and outside a building. Thus the use of UV-C sources installed inside a space or room itself has proven to be inefficient and largely ineffective. If one kills 80%% of microbes, the remaining will reproduce in the room more rapidly than one is killing them. Also convection currents in the room make it difficult to control air flow.

The result is that only a portion of air-flow is actually exposed to UV-C radiation, and the balance of UV-C energy is wasted This approach has proved unable to reliably treat all of the air in a room.

Overall, the difficulty in shielding people and materials from UV-C radiation and the inability to adequately treat large, rapidly-moving volumes of air in open spaces, has caused the use of UV-C light up to this time to be largely confined to the disinfection of water in closed containers, and to small volumes of air in holding enclosures, in the small area surrounding condenser coils or in closed areas that are unoccupied. There are also large, mobile units that bathe a room with UV-C light in an amount sufficient to effectively sanitize an area. However, in a hospital or healthcare facility, they can be used only when a room is unoccupied, for example between patients, due to the inherent danger of over=exposure of a person to UV-C radiation.

### C. Healthcare Applications

At least one embodiment of the invention can be used in a forced-air system and/or stand-alone air conditioner in virtually any building, room or area. However one application in particular is a priority for installation—a hospital, senior living or healthcare facility.

Hospitals in particular are in urgent need of the sterilization capabilities provided by the present invention. Hospital surgeries are ideally performed in a completely sterile operating environment, so that no post-operative inflammation, infection, or unintended tissue damage will occur in a patient. In addition, a patient staying in the hospital can be exposed to microbes in the air during their stay. However despite the best efforts of those in the medical profession, an estimated 0.5% to 10% of procedures result in surgical-site infections (SSI). That equates to over 275,000 patients in the United States each year. Individuals with an SSI have a mortality rate twice that of someone without an infection. A patient with an SSI stays in hospital, on average, one week longer than someone without an infection. In total, SSI cost the U.S. up to $10 billion a year in extra patient hospital costs. An estimated 8,200 deaths are attributed to SSI each year in the U.S. alone.

Efforts to reduce the number of SSI infections have had limited success. With the rise of drug-resistant bacteria, such as MRSA (methicillin-resistant Staphylococcus aureus), the problem shows no signs of slowing, and some scientists are concerned that the issue may continue to worsen.

It is an object of this invention to address deficiencies of known room air sterilizers and provide an air disinfection system and method that can be used pervasively throughout a facility. More particularly, it is an object of this invention to provide a novel, easily-installed, easily-maintained and low-cost method of safe and effective air disinfection for both retrofit and new installations.

### D. Definitions

SLS—The term “SLS” is used to refer to a “sterilization light source”, e.g. a source that produces light in the UV-C range. An SLS may be any of, for example e.g., a solid-state light source such as an LED, OLED, laser-driven source and/or any other type of light-producing solid state device. An SLS can also be a non-solid-state light source, such as e.g. electrochemical, discharge lamp (e.g. fluorescent tube, CFL, HID), HMI, induction lamp, excimer lamp, plasma lamp, incandescent lamp, halogen lamp, arc lamp or a combustion light source. An “SLS array” will include at least one UV-C sterilization light source. The terms “SLS”, “LED” and “UV-C source” are used interchangeably in the Specification.

TITHONIC—“Tithonic Rays” was the original name given by Prof. John William Draper in 1842 to the invisible rays he discovered in sunlight. Today, radiation in that portion of the electromagnetic spectrum (between 10 nm-400 nm) is officially termed “Ultraviolet”, and the frequency range is further divided into sub-classifications such as UV-A, UV-B, UV-C, near UVC and others as discussed herein.

UV-C DEVICE—The terms “UV-C Device”, “UV-C Module” or “UVC-Source” are used interchangeably in the specification to refer to an embodiment or implementation of an embodiment of the invention. This term is to be distinguished from a “UV-C Source”, which refers specifically to the UV-C light producing element that is generally contained within a UV-C device or UV-C module.

UV-C LIGHT ENGINE—The term “UV-C Light Engine” refers to a device containing a source of UV-C radiation included in an embodiment of the invention. The UV-C source may be one or more of an LED, mercury-vapor lamp, fluorescent lamp, excimer lamp, laser-driven source or other light source that produces UV-C radiation within the subject UV frequency range. UV-C light from a UV-C source in the Light Engine is collimated into a UV-C beam. Output of a UV-C Light Engine is optically-coupled to a means of directing the UV-C into the path of the airflow in the HVAC system.

WINDOW—in an exemplary embodiment of the invention referred to as Implementation 1 (see FIG. 1), the term “Window” refers to a section of UV-C resistant Glass or Polymer that is placed between a UV-C source located in the embodiment and an access opening in a duct to enable UV-C light from the embodiment to pass through the window and irradiate air flowing through the duct (see FIG. 1-3)

OPTIONAL ACCESSORIES—Exemplary Embodiments of the Invention may be equipped with optional compatible accessories that can be added to the UV-C Device in order to improve efficiency and/or add functionality, based on the location of the UV-C Device. These are particularly applicable where the UV-C device is ceiling-mounted. Optional Accessories can be attached to an embodiment and located above the ceiling line in a plenum space, and/or below the ceiling in a room. Optional Accessories include but are not limited to, for example e.g.:

Sensor—such as:
Air Quality Monitor
Camera
Microphone
Proximity
CO/CO₂
   Motion
   Smoke
   Particulates
   Gases
   Fluids, Water and Humidity

Communications—such as:
Wireless Transceiver
   e.g. IRDA, RF, ultrasound, LIFA
Alarm+Strobe
Speaker/microphone
Lighting control
Camera remote transmitter
Badge Reader

APPARATUS—Apparatus refers to a collection of elements that comprise the operating elements of a given embodiment or implementation of the invention.

DUCT—Duct refers to a section of the distribution system for the treated air produced by an HVAC unit. A duct may be of and cross-section, shape or material, and may be assembled from sections installed at the installation.

HVAC—HVAC is an industry term that stands for Heating, Ventilation and Air Conditioning. In this specification, HVAC is used to describe any system that provides at least one of these functions. It includes not only conventional HVAC, but also air treatment equipment such as a heat pump, geothermal air treatment, fans, convection systems and devices performing equivalent functions.

HELICAL BAFFLE/AIR FOIL MOUNTED IN DUCT OR PIPE—A transparent helical insert that is mounted inside of a tube or duct wherein the edges of the helical insert are sealed where they meet the sidewalls of the duct or tube, forcing air flowing through the duct to follow the entire length of the helical insert, thereby extending the length of the air path and consequently the time in which the air passing through the duct can be exposed to UVC radiation. The diameter of the helix may vary along its length depending on the method of manufacture, but the basic concept is to create a spiral air path. The helix may have a wider diameter at one end than at the other, or at any point in between. The outer edges of the airfoil will be sealed to the walls of the duct/duct insert, and also on the inside edges, forcing all the air to go through the entire length of the air path. UVC radiation is provided by one or more UVC sources placed inside a tube that runs through the center of the helical baffle, as shown in FIG. 18-23. There may be additional similar tubes that extend through one or more of the baffles, as shown in FIG. 19, in which one or more UVC sources can be installed. The intersection of these smaller tubes and the helical baffle are also sealed, so that all air moving through the duct is forced to follow the helical path.

IMPLEMENTATION—An implementation is an exemplary embodiment that has specific features that are configured to be appropriate for a specific type of use or of customer. The terms Implementation and Exemplary embodiment are used interchangeably in the specification. Description of an implementation is included to aid in the understanding of the application of the invention to a particular situation, and not in any way to limit the claims for the invention.

STERILTIZE—The words “sterilize”, “sanitize”, “purify”, “cleanse”, “disinfect” “anti-microbial”, “decontaminate”, “microbial reduction” and similar terms are used interchangeably in this application. The intended meaning is that exposure of a given fluid to UV-C in an embodiment of the invention is sufficient to remove enough of a target contaminating organism so that the fluid is measurably free of the target contaminant to meet or exceed a specific standard.

FLUID—A fluid is a substance that flows, including liquids, gasses, plasmas, plastic solids like Silly Putty. More specifically, a fluid is a substance that continually deforms under applied shear stress over any period of time. This characteristic of a fluid is referred to as its viscosity.

### BRIEF DESCRIPTION OF THE DRAWINGS

FIGS. 1A and 1B illustrate an embodiment mounting UV-C Apparatus on the outside of duct; 1A using UV-C LED lattice and 1B using linear UV-C discharge lamps (e.g. UV-C fluorescent or HID lamp).

FIG. 2 illustrates details of Internal configurations of the Embodiment shown in FIG. 1.

FIG. 3 illustrates an exterior perspective drawing depicting external mounting of the embodiment shown in FIG. 1.

FIG. 4A illustrates a top view of a UV-C lattice mounted internal to the duct details.

FIG. 4B illustrates a side and end view of a UV-C lattice mounted internal to the duct.

FIG. 4C illustrates details of UV-C LEDs installed on a linear strip mounted internal to the duct.

FIG. 4D illustrates details of UV-C LEDs installed on a linear strip mounted internal to the duct.

FIG. 5 illustrates an embodiment integrated into standard ceiling tile to treat the return air in plenum. A number of accessories are also indicated on the drawing.

FIG. 6 illustrates an embodiment configured to “piggy-back” on top of a conventional light fixture in a ceiling plenum with optional accessories.

FIG. 7 illustrates an embodiment with UV-C Apparatus installed on top surface of air register above plenum also indicating optional accessories for data collection, communications, alarms and controls.

FIG. 8 illustrates an embodiment with UV-C apparatus built directly into a diffuser.

FIG. 9 illustrates an embodiment with UV-C sources mounted inside a custom low-profile diffuser enclosure.

FIG. 10 illustrates an embodiment with UV-C apparatus built into collar installed between duct and diffuser below ceiling line.

FIG. 11 illustrates an embodiment of UV-C apparatus with Slide-in modules for installation in duct-diffuser joiner collar above ceiling line.

FIG. 12 illustrates an embodiment for use with window air-conditioner, heat-pump.

FIG. 13 illustrates an embodiment of UV-C Apparatus with small footprint unit mounted in multi-layer stack.

FIG. 14 illustrates an embodiment of UV-C System with computer controlled baffles, helical inserts, UV-C lattice.

FIG. 15 illustrates an embodiment of computer-controlled array of baffles.

FIG. 16 illustrates an embodiment describing detail of UV-C lattice, computer-controlled array of baffles (end-caps), and tube assembly showing typical placement of helical insert, UV-C lattice and computer-controlled baffles.

FIG. 17 illustrates an embodiment describing detail of a single duct containing UV-C lattice array and helical insert.

FIG. 18 illustrates an embodiment describing detail of a single duct containing a linear tube that houses at least one UVC source consisting of a fluorescent UV-C tube or similar discharge UVC source, or a linear LED array, or combination of UVC sources, and where the linear tube penetrates through multiple layers of the helical insert. The junction between the tube and each layer of the helical insert is sealed around the outside of the tube.

FIG. 19 illustrates an embodiment similar to that described in FIG. 18 with one or more additional tubes containing one or more additional UVC sources, where each tube penetrates one or more levels of the helical baffle to further increase the amount of UVC to which the moving air is exposed.

FIG. 20 illustrates an embodiment where a UV-C light engine produces a concentrated collimated beam optically coupled to a side-emitting light pipe made from a material that does not degrade significantly in UV-C and extends the path of the UV-C radiation.

FIG. 21 illustrates an embodiment of UV-C Apparatus optically coupled to a UV-C side-emitting Light Pipe.

FIG. 22 illustrates an embodiment of UV-C Light Engine coupled to light pipe and supported by adjustable stand-offs.

FIG. 23 illustrates an embodiment of UV-C Apparatus with light engine optically coupled to light pipe mounted in helical airfoil.

FIG. 24 illustrates an embodiment of UV-C apparatus into a medical Instrument to deliver UV-C using fiber-optic inserted in small-diameter hollow needle (e.g. acupuncture-size) to specific local site.

FIG. 25 illustrates an embodiment of UV-C apparatus integrated into an HVAC diffuser with UV-C source mounted on top of each diffuser level and an Ambient Light source mounted on the bottom side of the diffuser level, so that the entire assembly operates as a lighting fixture as well as a UV-C sanitizing air diffuser. Ambient light sources may produce colors of light (including white) that can be remotely-controlled and programmed using a microcomputer. The UV-C Apparatus may also include one or more communicating sensors and controls.

### DETAILED DESCRIPTION

Capabilities of the UV-C sterilization apparatus embodiments described herein include, but are not limited to, one or more of the following:

(1) Uses direct exposure to UV-C to disinfect a fluid, without photochemical steps or electrostatic treatment, by creating an extended path within a delivery system for the fluid by means of which the exposure time of the fluid to the UV-C radiation is extended sufficiently to kill micro-organisms in the fluid that are dangerous to humans.

a. Where the effectiveness is further enhanced by the addition of an electrochemical and/or electrostatic disinfecting agent

(2) disrupts the DNA of the target microbes to effectively kill them;

(3) Shields UV-C light so that UV-C sensitive organs (such as human eyes) and UV-degradable materials are not exposed for repeated or prolonged periods;

(4) maximizes the exposure of the fluid stream to UV-C light;

(5) enables self-cleaning of the UV-C source to minimize maintenance

(6) enables maintenance and upgrading of UV-C technology and components using simple, plug-in modules (for example e.g. the potential substitution of “far UV-C” sources in the 200 nm range in future);

(7) is compatible with existing construction techniques, products, codes and practices, so that standard calculations, designs, equipment and techniques may be utilized to design and install the system;

(8) will not significantly compromise the efficiency or performance of an existing fluid distribution system;

(9) utilizes a readily accessible source of power in the area of the installation, or if no external power is available, to be powered by a local source such as a battery

(10) is easily installed with conventional ducts, tools and installation techniques

(11) is adaptable to different sizes, types and lengths of duct and diffusers.

### A. Overview

The invention includes a multiplicity of possible implementations, depending on the characteristics of a fluid and the microbes to be eradicated. At least one application is to provide a means of air sterilization in forced-air ventilation systems. Such embodiment places a UV-C light source in a location where the UV-C radiation is shielded from human occupants, while exposing the air moving through a forced-air ventilation system to an amount of UV-C radiation for a sufficient period of time to sanitize the air.

Examples of locations where an embodiment of the invention can be installed for forced air sterilization include, but are not limited to: a ceiling plenum, on top of a conventional ceiling-mounted light fixture in an area above the ceiling line (piggy back), integrated into a conventional drop-in ceiling panel, and/or inside an element of a forced-air ventilation system, including but not limited to, ducts, plenum return paths, vents and/or diffusers, as illustrated in the drawings included herewith of exemplary implementations.

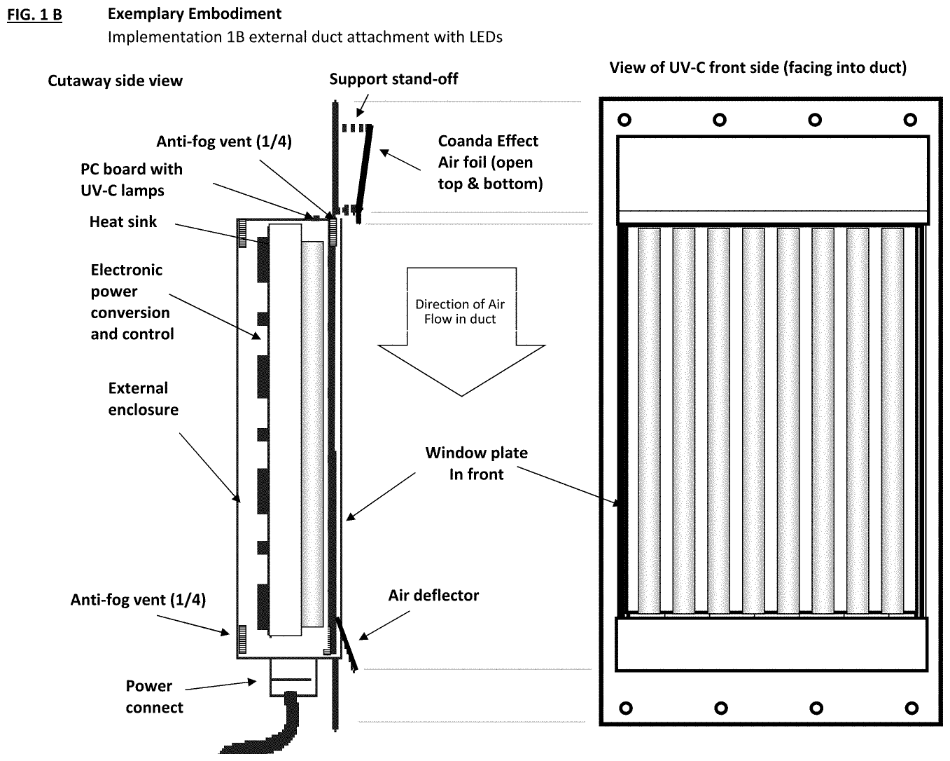

Power for an embodiment may be accessed by a connection to a nearby junction box, typically containing a non-dimmed lighting circuit, or to an available low-voltage circuit for lighting, thermostat, ventilation or other devices. In some cases, a cable may be “home run” to a local circuit breaker, power panel or remote power supply, in accordance with applicable codes. A power feed may include a transformer or LED driver, and/or a battery-pack, as shown in FIG. 2A-C. Since the methods of electrical connection are well-known to those skilled in the field, these are not as such the subject matter of the claims in this invention.

### B. Exemplary Embodiments and Implementations

An implementation of the invention is an embodiment that has been configured to meet the demands of a specific application, in response to installation, budget, environmental or other requirements. The embodiments and implementations described herein are mentioned not to limit or define the invention, but to aid understanding thereof. Exemplary embodiments are discussed in the specification, and further description of the invention is provided there. Advantages offered by the various embodiments and implementations of the present application may be further understood by examining this specification.

Other features, advantages and configurations of the present invention will become apparent to one skilled in the art upon examination of the detailed description together with the accompanying drawings. It is intended that all such features, advantages and configuration be included herein as embodiments within the scope of the present application and protected by the claims thereof.

Seven types of implementation are described as exemplary embodiments in the Specification and drawings herein. These are some, but not all, possible implementations of embodiments of the invention, and may include additional accessories as described herein. The exemplary embodiments are included solely as examples to aid in understanding and implementation of the invention. These and other variations of these exemplary embodiments are included in the spirit of the invention and covered under the claims.

Implementation 1—Unit for Installation on the Outside of an Air Duct

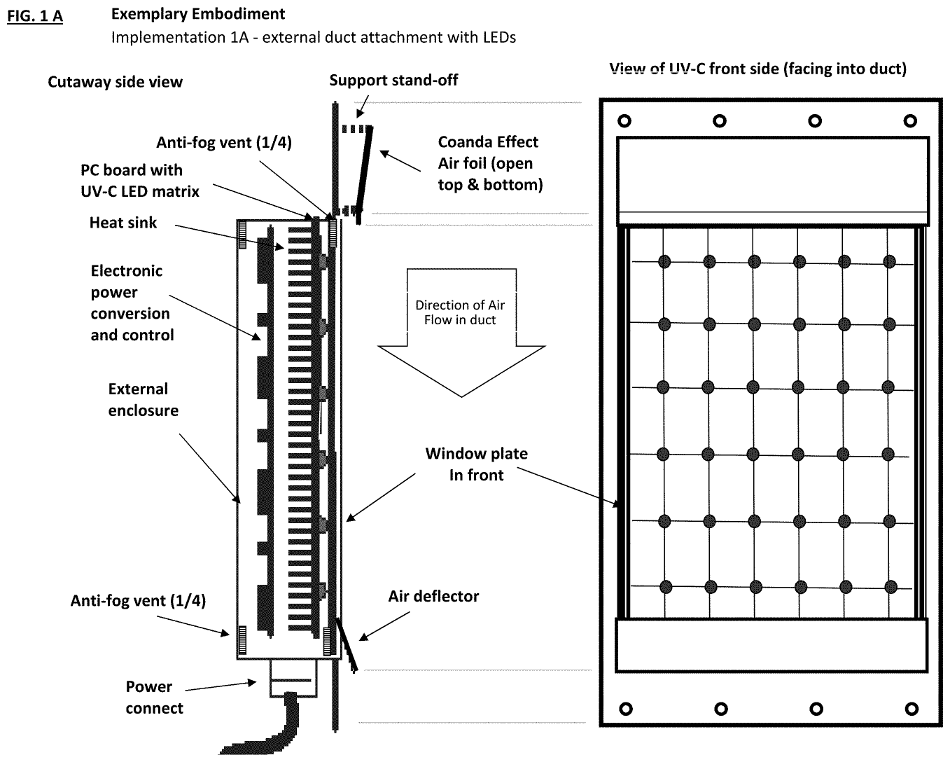

As shown in FIG. 1-FIG. 3, an Implementation of the invention is packaged in an enclosure designed so that at least one instance of the invention is inserted with the UV-C output facing through a window in the duct. The assembly comprising the invention is mounted into an appropriately sized opening in the side of a conventional HVAC duct in such a manner that the air passing through the duct is exposed to UV-C radiation in order to purify the air (see FIG. 1-3). Depending on the size and shape of the duct, multiple UV-C units may be installed, arranged all on one side of a duct and/or on more than one side of a duct. In an existing forced-air system, where the invention is installed as a retrofit, an opening of the appropriate size is cut into at least one side of the duct and the device is inserted into the hole and fastened; the edges are then sealed using conventional metalized duct tape or similar sealing method used in the trade. This exemplar embodiment applies to both duct installation where an opening is cut into a section of duct to provide access to the airflow, or the invention is pre-installed into a joiner section of duct that is inserted between other duct sections.

Implementation 1 of the invention is packaged in a metal enclosure as shown in FIG. 1-3. An opening of the appropriate size is cut into a duct. A UV-C device is positioned over the opening and mounted on the outside of the duct so that a glass or UV-C resistant “window” on side of the UV-C device that faces into the duct (“front” of the UV-C unit). Inside the enclosure of implementation 1, a circuit board and heat-sink assembly containing at least one UV-C source is mounted so that the light from the UV-C source shines through the window into a duct to irradiate air passing through the duct. The window is mounted so that it is essentially flush with the side of the duct.

Inside implementation 1, an air foil that uses the Coanda effect can be located along the top edge of the window in order to create planar airflow at accelerated speed that flows across the surface of the window plate to keep it clean and prevent fogging. The bottom edge of the device has a deflector that pushes the planar sheet of air back into the primary airstream. In addition, one or more antifogging vents in a UV-C Apparatus enclosure will cause air from the duct to enter inside the invention enclosure behind the window in the direction of the airflow in the duct and to exit on the other end, driven by both convection and the air-pressure differential created by the Coanda foil. Air from inside the UV-C apparatus can also flow into the duct. This equalizes the temperature in the UV-C apparatus and duct to prevent condensation on the window that could lessen the amount of UV-C injected into the duct.

In one implementation of the embodiment, for example e.g. in a new HVAC installation, the invention can be delivered completely pre-assembled into a section of joiner duct containing the UV-C sources and other elements of the invention as described herein. The joiner duct is inserted in between sections of conventional duct as the system is being installed and connected to a convenient source of power. This can reduce the time required to install the UV-C apparatus.

Implementation 2—for Internal Installation Across a Duct with Airflow Through a UV-C Lattice

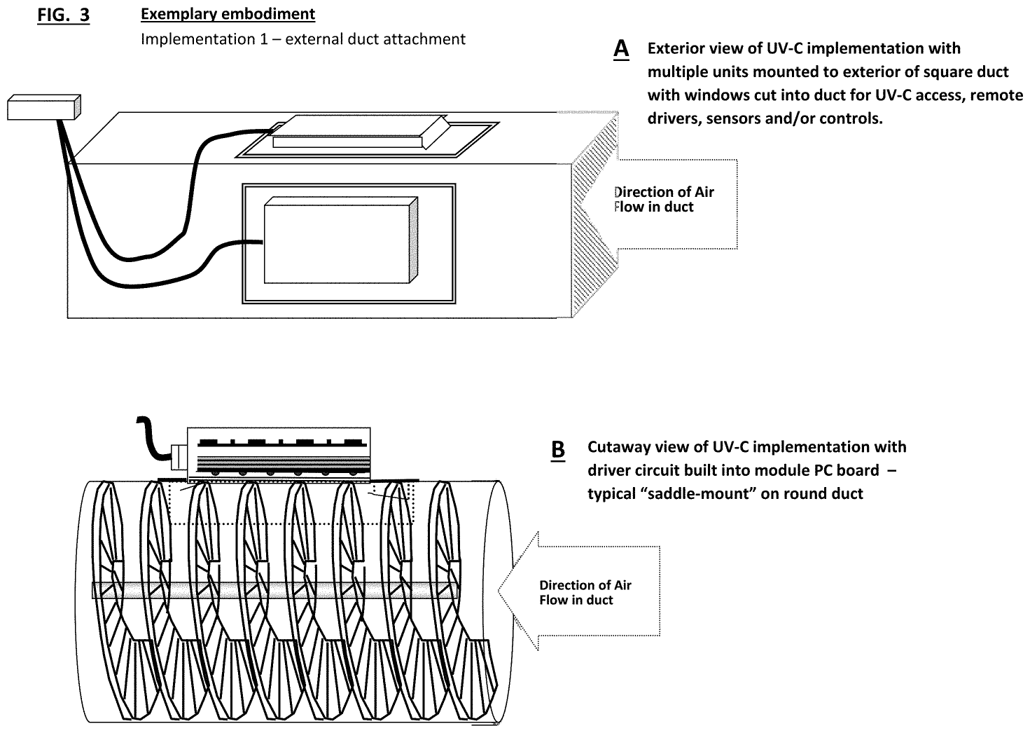

In at least one embodiment one or more UV-C sources are mounted in an open lattice configuration that is placed internal to the duct so that air moving through the duct passes through the UV-C Lattice module, as illustrated in FIG. 4. In this implementation, the rear of the lattice, opposite the UV-C sources, may include blades on the downwind side of an SLS and joined with heat-conductive material to act as a heat-sink for the SLS. On the other side of the SLS, facing into the air stream, a small air-funnel whose spout is approximately the same size as the UV-C chip may be mounted just in front of the UV-C chip to focus a narrow jet of high-velocity air on the front of the SLS to keep it clean as shown in FIG. 4.D. An air funnel should be made of a transparent UV-resistant material such as clear glass, silicone or polycarbonate to minimize a shadow cast by an air funnel.

Implementation 3—UV-C Integrated into a Standard Drop-in Ceiling Panel Size Enclosure

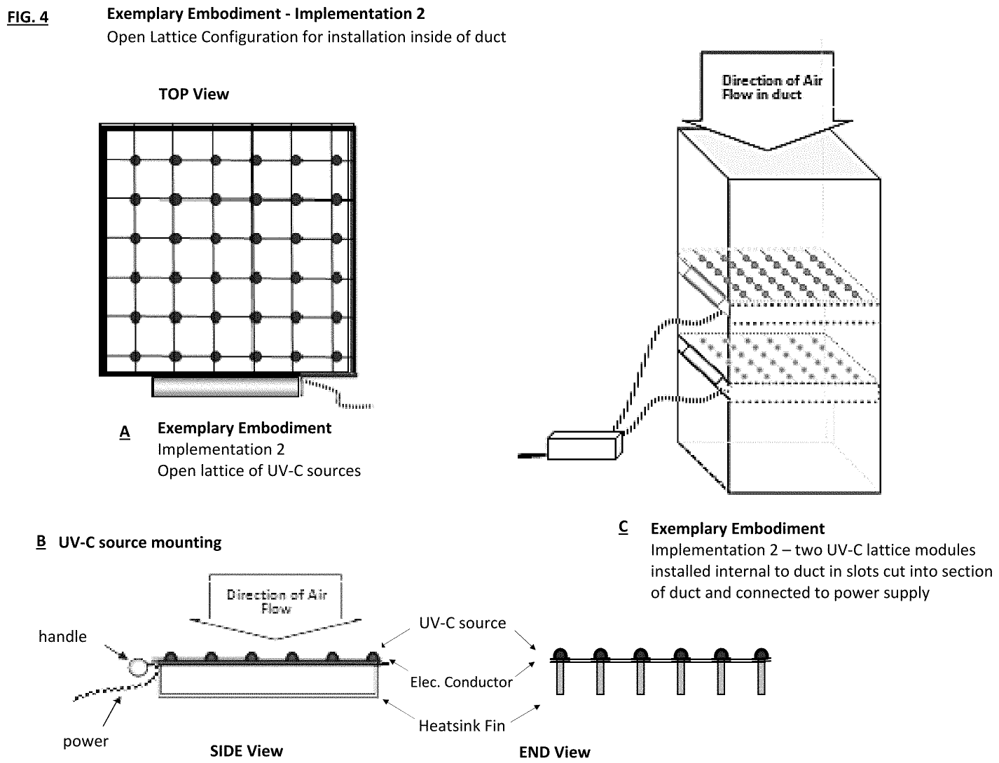

At least one embodiment integrates the elements of the invention into a UV-C mounting panel whose dimensions enable it to fit into a standard opaque dropped ceiling panel space. The UV-C source array is located on the top of the UV-C mounting panel so that all of the UV-C radiation goes into the plenum space above the dropped ceiling. The plenum is often used for the return path of a forced-air ventilation system, so that the return air can be purified before it reenters the system. An exemplary configuration is shown in FIG. 5.

Implementation 4—Collar Adapter for a Diffuser

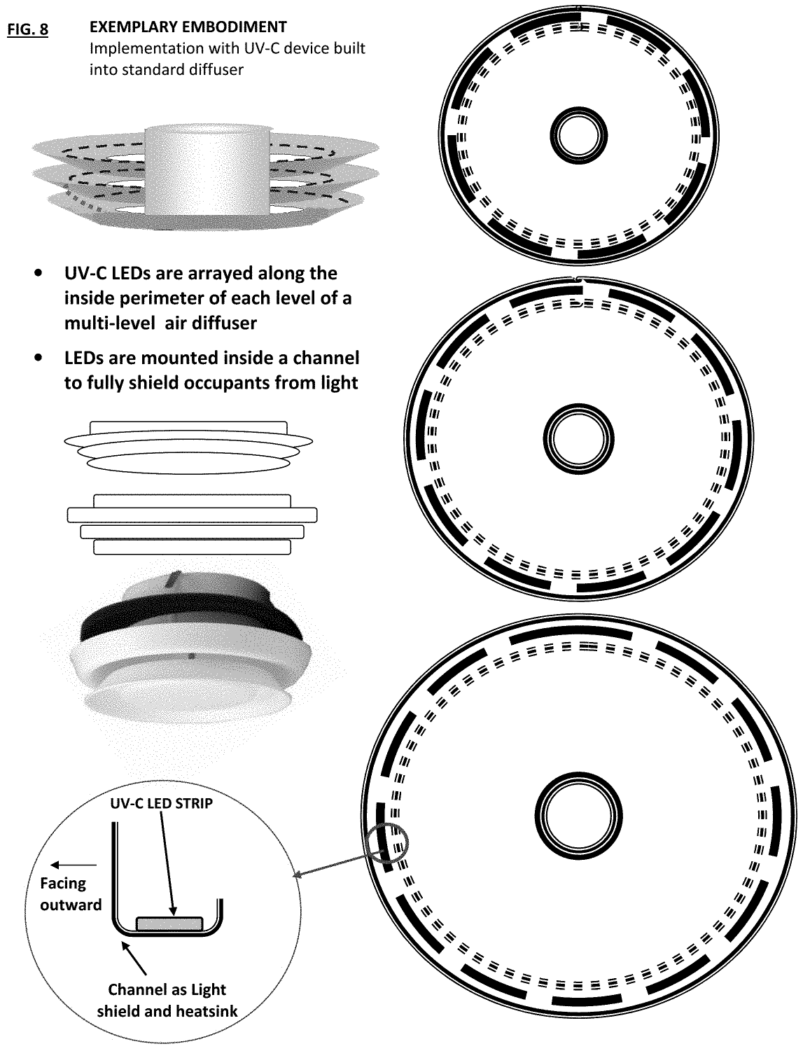

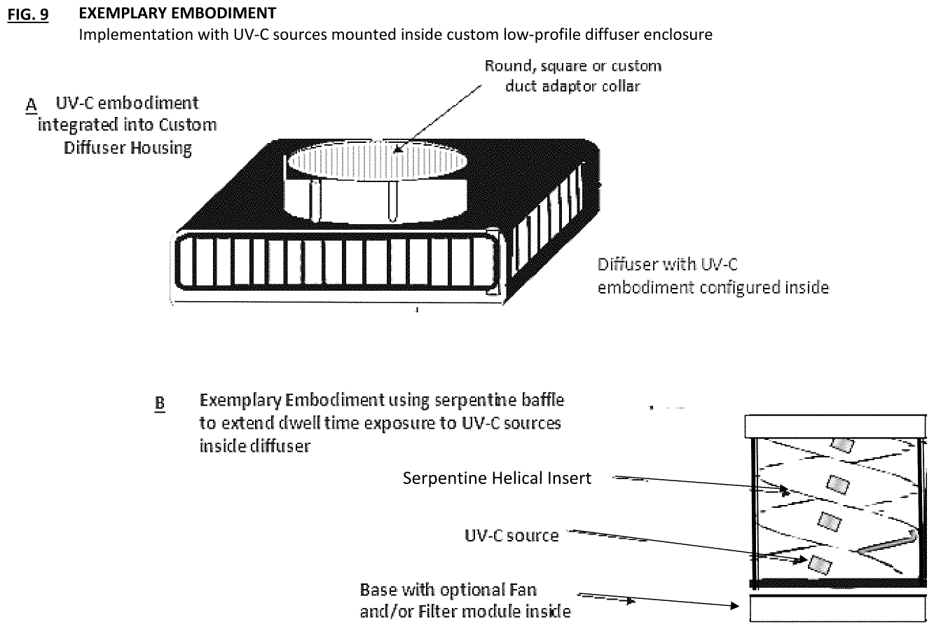

In exemplary implementation 4 as shown in FIG. 10-11, the invention is packaged in a “collar” that can be installed as a “joiner” between a conventional duct and a diffuser. The collar may be mounted either above or below the ceiling line. In an exemplary embodiment, when the invention is packaged in a diffuser collar above the ceiling line, as depicted in FIG. 11, it is applicable to spaces with relatively low ceilings.

Implementation 5—UV-C Source Built into a Diffuser

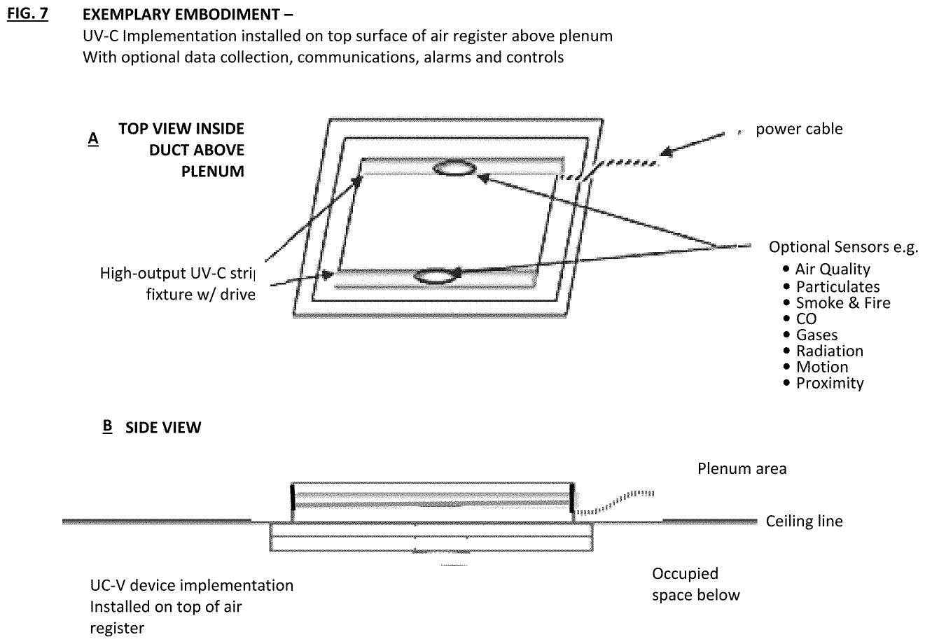

In exemplary embodiment implementation 5 the invention is packaged so that the embodiment of a UV-C air sterilization apparatus as described in the specification are installed in a custom diffuser housing such as shown in FIG. 9 that is designed to be attached to a conventional duct using standard installation methods.

Implementation 6—UV-C Apparatus for a Window-Mounted or Portable Air Conditioning Unit

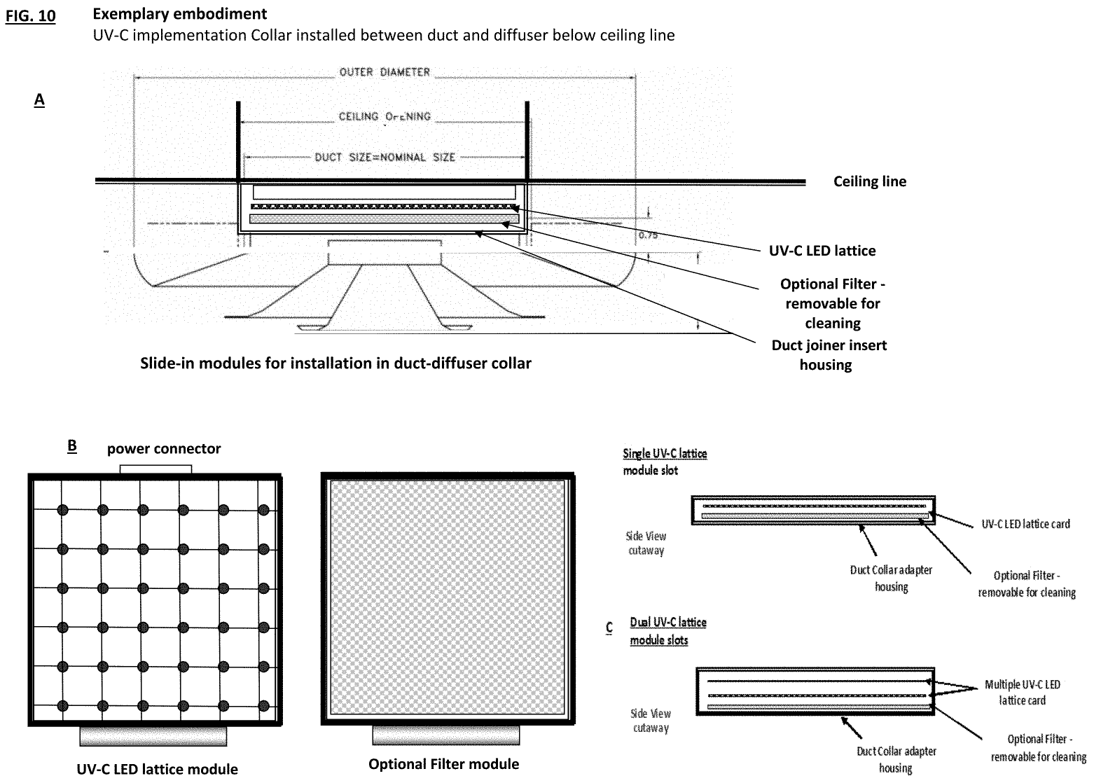

In this embodiment the UV-C apparatus is configured in a frame that can be installed into a window air conditioner, heat pump or similar device as illustrated in FIG. 12. The UV-C sources are mounted in a channel that is reflective on the inside to direct the UV-C radiation into the airstream coming out of the air handling unit while shielding those near the unit from UV-C exposure. A heatsink is mounted on the rear of the UV-C source conductor to remove heat away from the SLS array.

Implementation 7—Helical Insert to Extend Air Path and Increase UV-C Exposure in a Duct

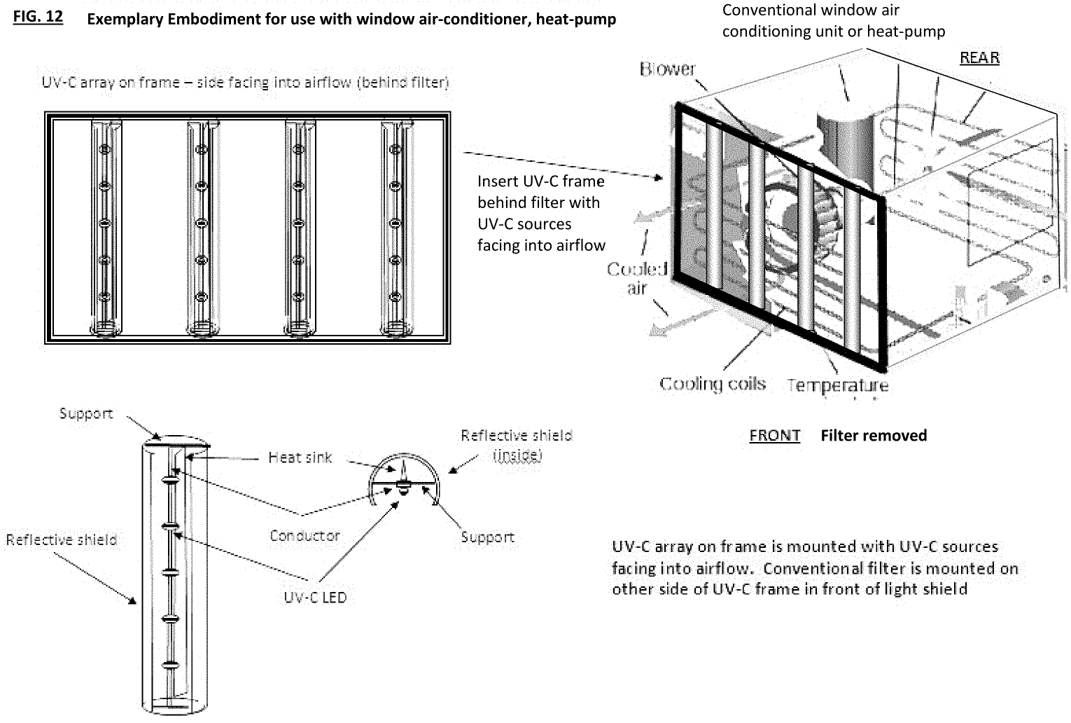

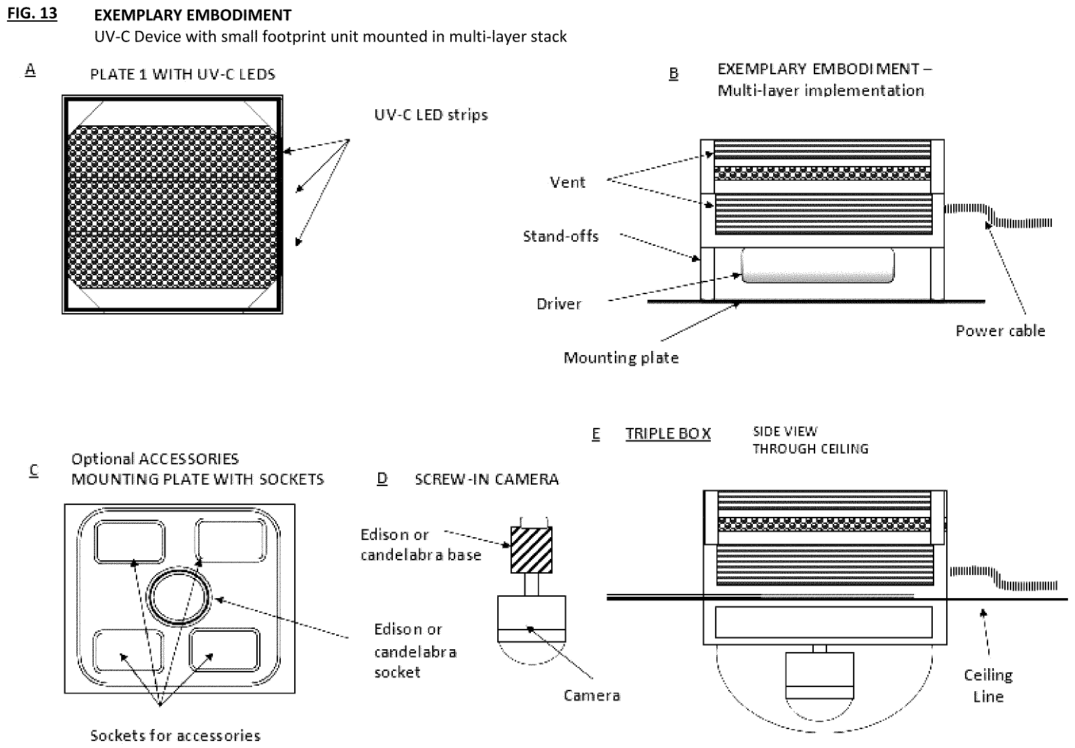

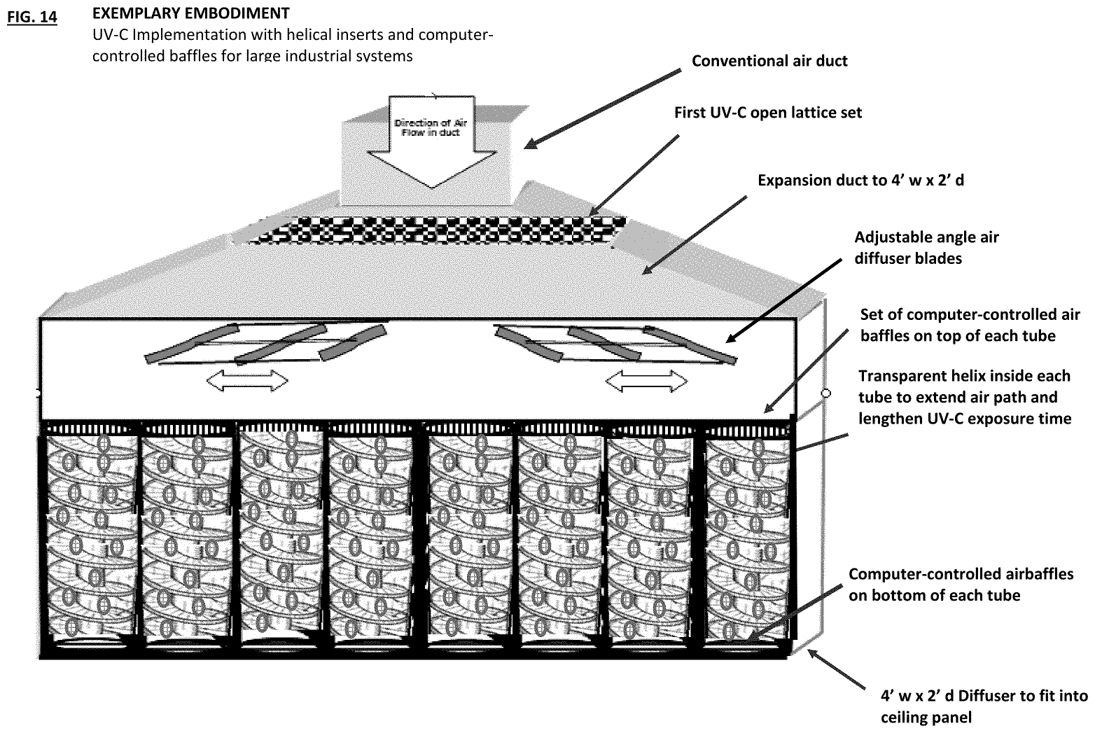

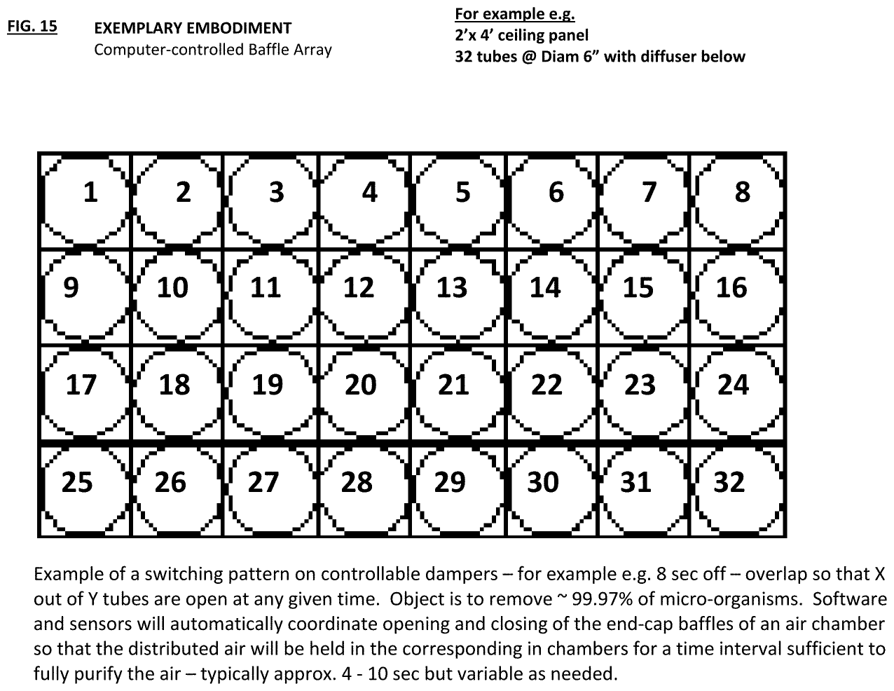

In at least one embodiment, a helical insert (air foil) made from a UV-C transparent material, such as polycarbonate or silicone, that may be mounted in a tubular structure, that is inserted into a duct in order to extend the length of the air flow path and consequently the time that air remains in the duct, in order to increase the UV-C exposure of the air as it flows through the duct. One or more UV-C sources are mounted in the duct positioned so as to irradiate the air passing through the helical insert. The UVC sources may be one or more open lattice arrays, as shown for example in FIG. 14-16, where the UV-C array is placed internal to the duct so that air moving through the duct passes directly through the field of UV-C radiation generated by the UV-C lattice. The density of UV-C sources in the array permits all of the air flow to pass in close proximity to a UV-C source. One or more UV-C lattices are inserted vertically into the levels of the helix to provide sufficient UV-C radiation to purify the air passing through the helical pathway. In at least one embodiment, as shown in FIG. 14, a duct is subdivided into one or more downstream ducts, wherein a downstream duct can have a set of computer-controlled baffles (or end-caps) at each end that can be opened and/or closed progressively in order to hold air inside a duct and further prolong exposure time of the enclosed air to UV-C radiation. Such computer-controlled baffles can be opened and closed in a coordinated sequence so that air that has been enclosed in at least one duct for a specified period of time is released by the opening of the computer-controlled baffles, while the computer-controlled baffles in at least one other duct are closed in order to provide extended exposure to UV-C in that second duct. This process of opening and closing the computer-controlled baffles is cyclically repeated so that all of the air flowing through the system progressively receives extended exposure to UV-C radiation, while delivering a flow of air into the space sufficient to maintain comfort.

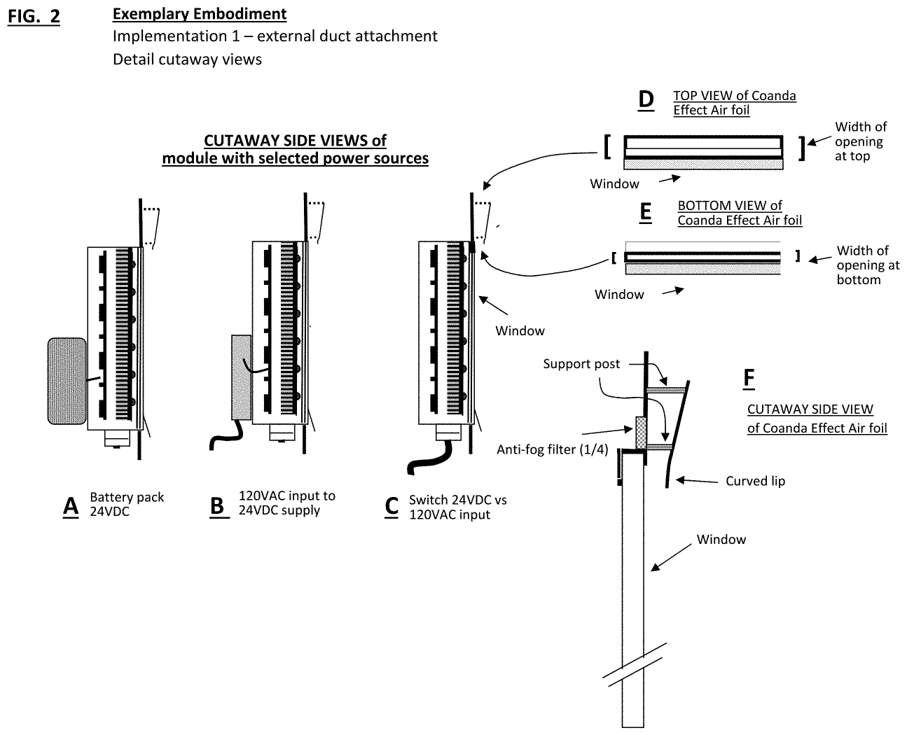

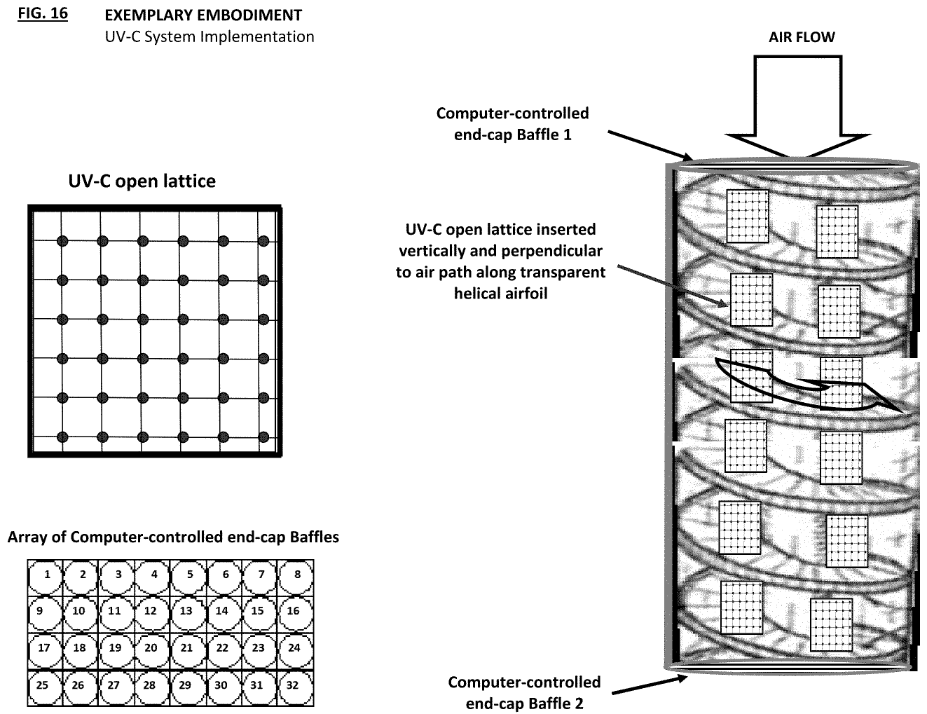

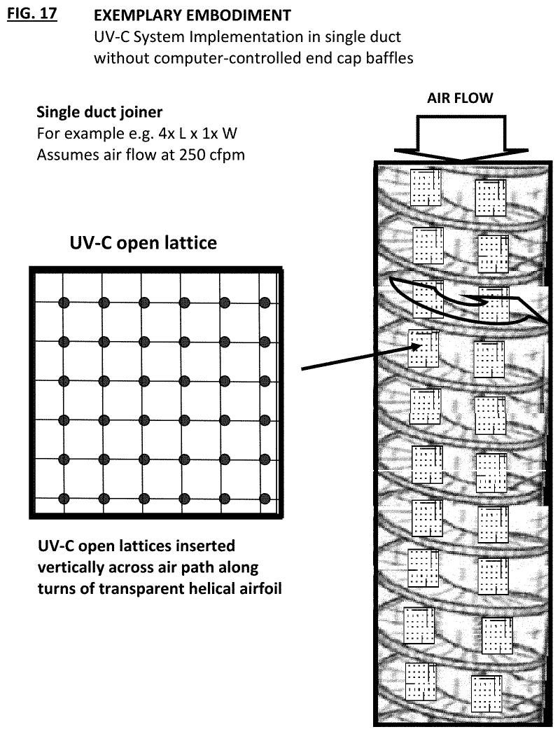

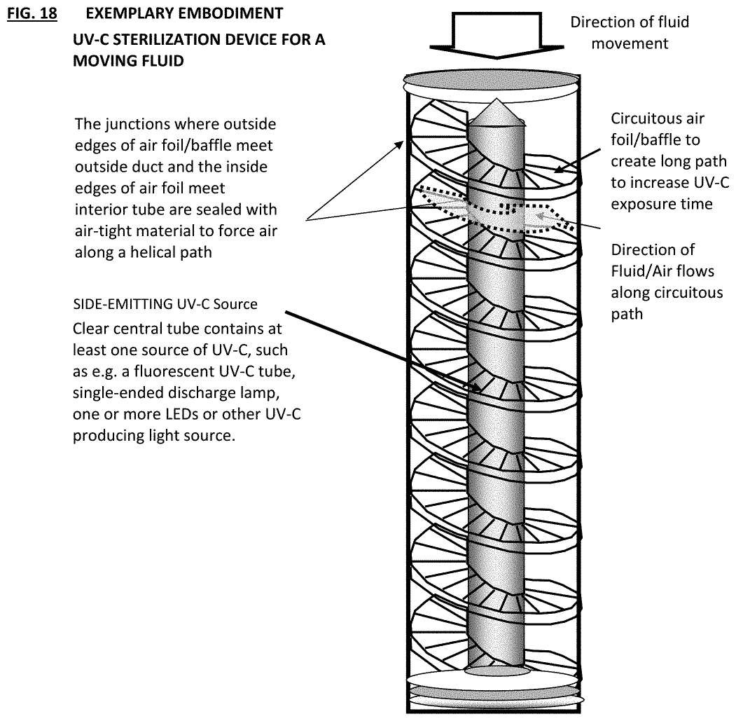

Finally, in at least one embodiment of the invention, instead of, or in addition to, a lattice of LED UV-C sources, one or more UV-C discharge lamps can be used to irradiate the air flowing through the helical transparent insert or airfoil. The UV-C discharge lamps may be inserted into a transparent tube or channel running through the center of the helical airfoil as shown in FIGS. 18 and 19 for example, or located outside of the helical airfoil to irradiate air passing through the airfoil, as shown for example in FIGS. 1A and 3B. Whatever the placement, a sufficient number of SLS UVC sources will be installed to generate a UVC field that provides enough UVC radiation throughout the length of the section of duct to fully irradiate the air passing through it and destroy any target microorganisms. If needed, additional clear tubes containing one or more additional UVC sources can be installed and sealed on the outside edges of the helical baffle, or in other locations penetrating the helical baffle and also sealed in such a way that the amount of UVC radiation can be further increased while air is forced to flow the length of the helix.

In at least one embodiment, the tube containing the helical insert is made from a flexible material, such as UV-C resistant silicone. The tube and the helical insert inside may be adjusted in length as needed for installation. It may also be substantially collapsed linearly to reduce the tube length in order to ship in a smaller container, then expanded to be installed.

In at least one embodiment, the helical airfoil assembly and UV source is built in a rigid housing that is designed to be fastened inside of a standard ventilation duct.

In a further embodiment, the helical airfoil and UV source are built into a rigid housing that is mounted onto an adjacent duct as a joiner between sections.

In a further embodiment, the helical airfoil assembly and UV-C source are incorporated into the output section of the HVAC unit itself as an integrated product.

In a further embodiment, the helical airfoil assembly and UV-C source are mounted in an enclosure that is designed as an accessory to attach to an HVAC unit, typically to be installed between the output of the unit and the first section of duct.

Implementation 8—Use of Side-Emitting Light Pipe to Extend Time of Exposure of Air to UV-C Light

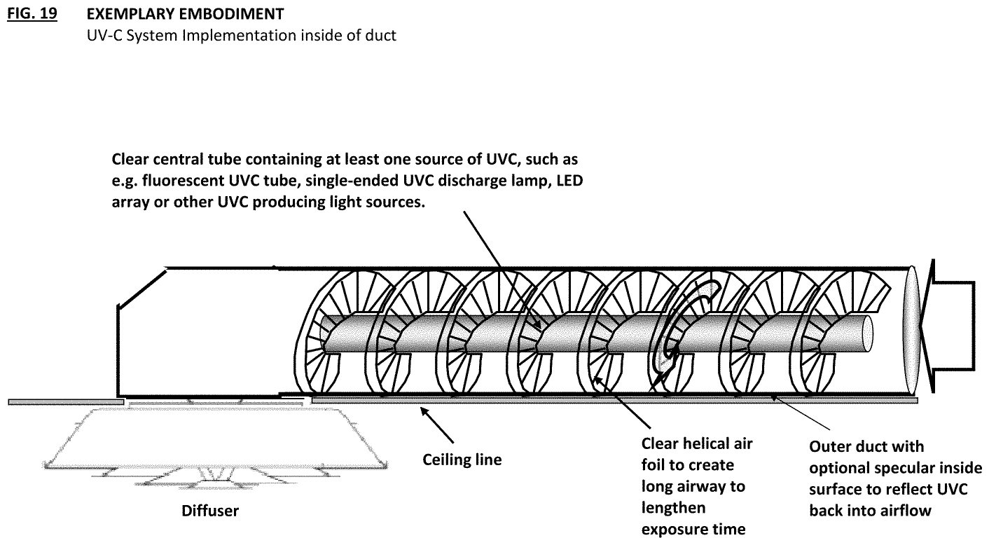

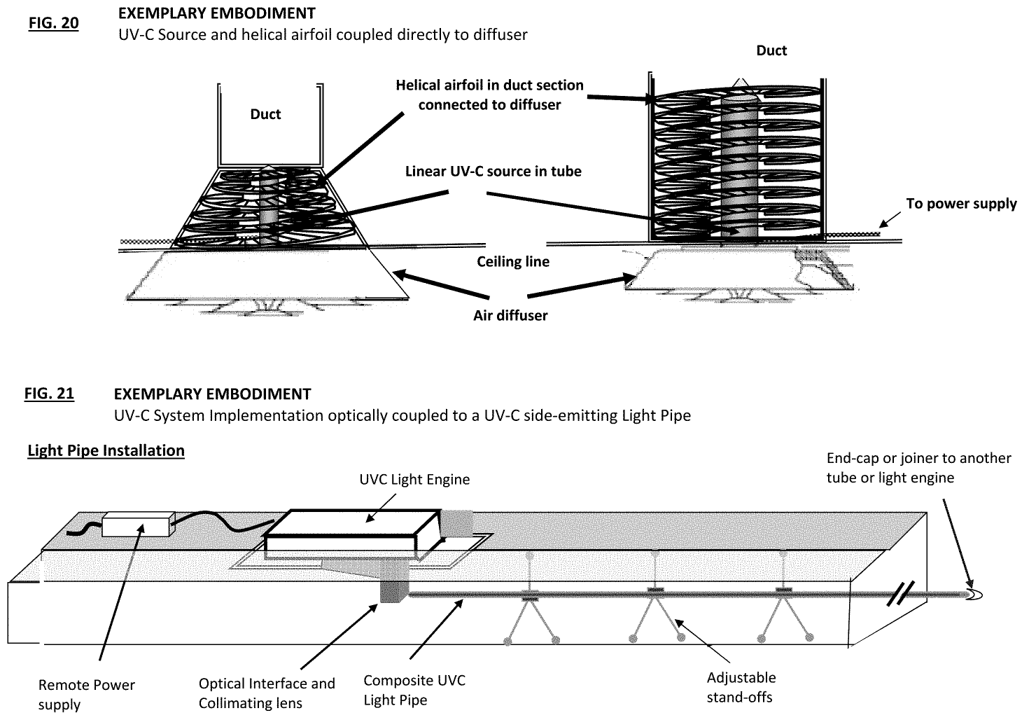

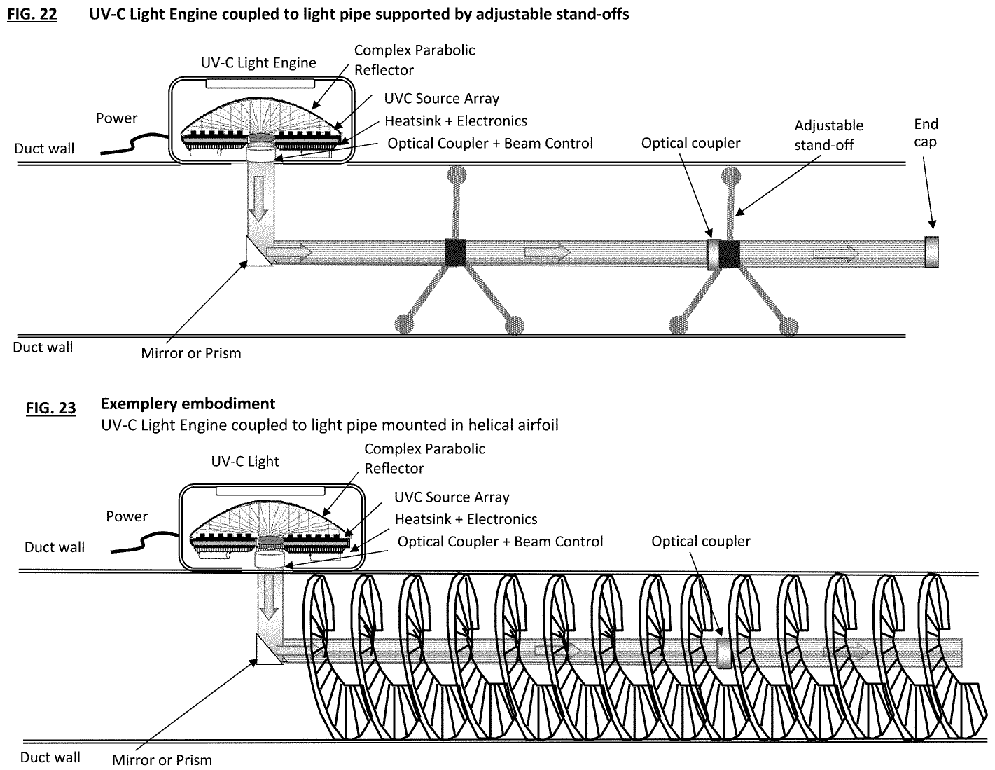

In at least one embodiment, as illustrated in FIG. 21-23, output of a UV-C Light Engine is collimated into a narrow beam. In at least one embodiment, this is accomplished using a suitably deep complex-parabolic reflector engineered to accommodate the size, shape and output characteristics of the UV-C source in the assembled Light Engine in order to maximize the output and focus. The output of the UV-C Light Engine can be further focused using a collimating lens appropriately placed near the focal point of the UV-C light source and reflector assembly.

In at least one embodiment, the collimated output of a UV-C light engine is optically coupled between the light engine and a length of side-emitting light-pipe or fiber optic. The coupler may include a mirror, prism or fused quartz rod used to direct the UV-C beam into the light pipe. The coupler may also serve to further collimate the UV-C beam.

In at least one embodiment of the invention, a light pipe is constructed as a solid or hollow length of light-conductive material that is able to withstand UV-C radiation in the target range of 200 nm-300 nm without significant degradation over time. Materials that meet this criteria at present include fused quartz, a glass slurry or various laboratory-grade glass (e.g. BK-7 material). Other suitable materials may be developed in future and employed without altering the intent of the invention. The light pipe will be side-emitting, in that the sides of the light pipe partially reflect light back along the length of the tube, while allowing a portion of the UV-C light to “leak” out of the sides of the tube all along the way until a point is reached where the attenuation of UV-C light become so large that the amount of UV-C radiation delivered is no longer effective for sanitization. The light pipe may be cut at a point, polished, and a reflective coating applied to reflect UV-C light traveling down the light pipe back towards the other end of the tube.

In at least one embodiment, a helical insert (air foil) made from a UV-C transparent material, such as polycarbonate, silicone, fused quartz, glass slurry or a comparable material or composite, that may be mounted in a tubular structure wherein the inner and outer edges of the airfoil are sealed to the outer and inner edges of the airfoil are sealed to the inner and outer tubes to form an assembly that is inserted into a duct in order to extend the length of the air flow path and consequently the time that air remains in the duct, in order to increase the UV-C exposure of the air as it flows through the duct. One or more UV-C sources are mounted in the duct positioned so as to irradiate the air passing through the helical insert. The UVC sources may be one or more open lattice arrays, as shown for example in FIG. 14-16, where the UV-C array is placed internal to the duct so that air moving through the duct passes directly through the field of UV-C radiation generated by the UV-C lattice. The density of UV-C sources in the array permits all of the air flow to pass in close proximity to a UV-C source.

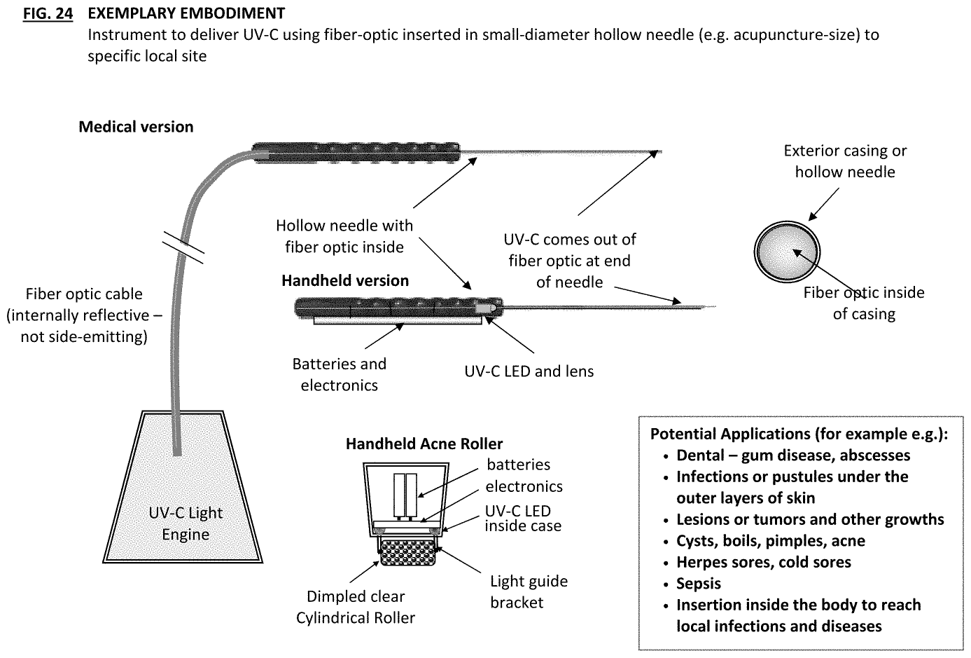

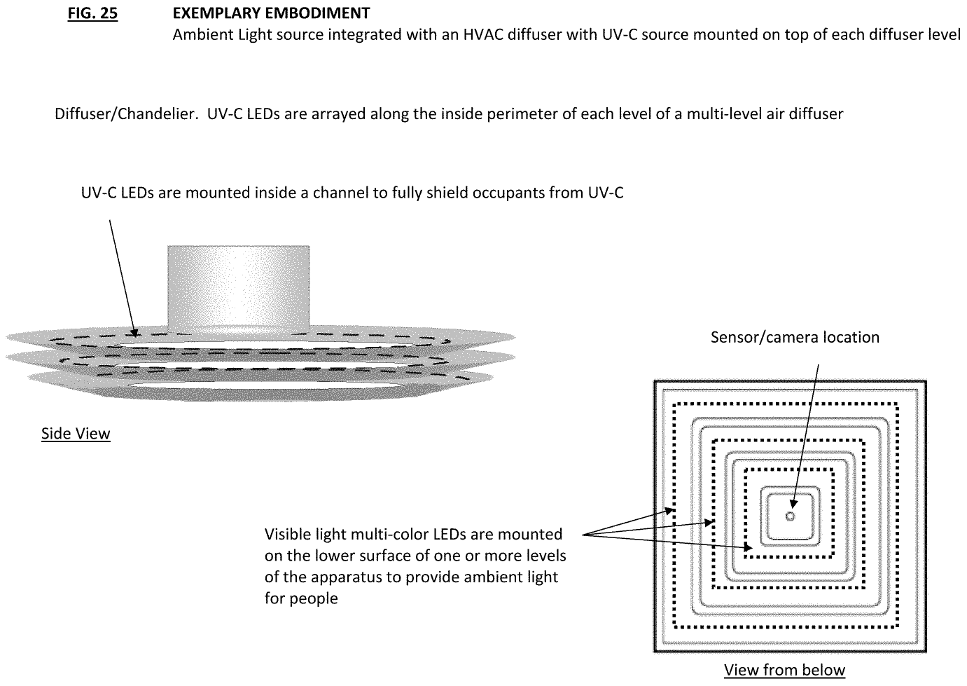

In at lease one embodiment, the collimated output of a UV-C light engine is optically connected to one end of a fiber-optic light pipe. In this embodiment the fiber optic is of a small diameter such that the opposite end where the UV-C light will exit is encased in a small hollow tube or needle with an outer diameter that approximates the size of an acupuncture needle, as depicted schematically in FIG. 24. The needle may be inserted into the body or under the skin of a patient to deliver UV-C light directly to the location of a local infection, cyst or tumor. The UV-C light is used to kill the infected or malignant cells in that specific area. In at least one such embodiment, a fiber optic is not side-emitting but entirely internally-reflective so that it releases UV-C light from the tip only.

### Accessories for Exemplary Embodiments

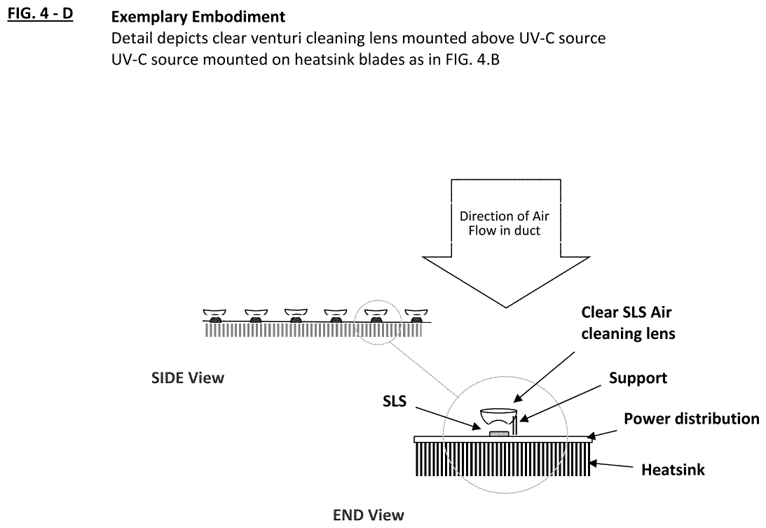

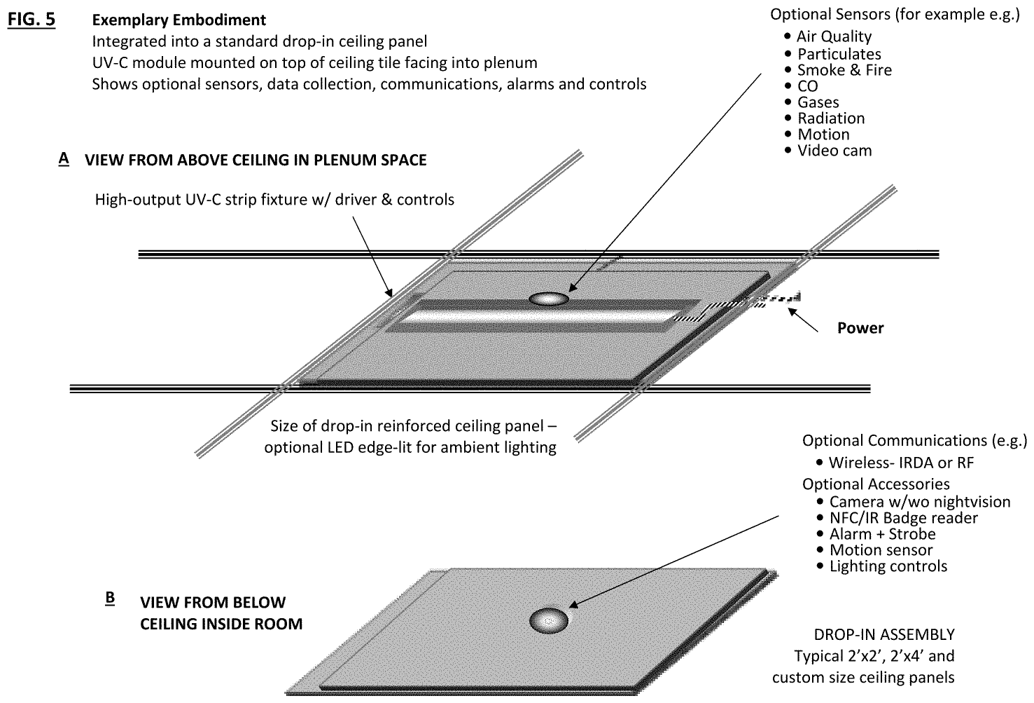

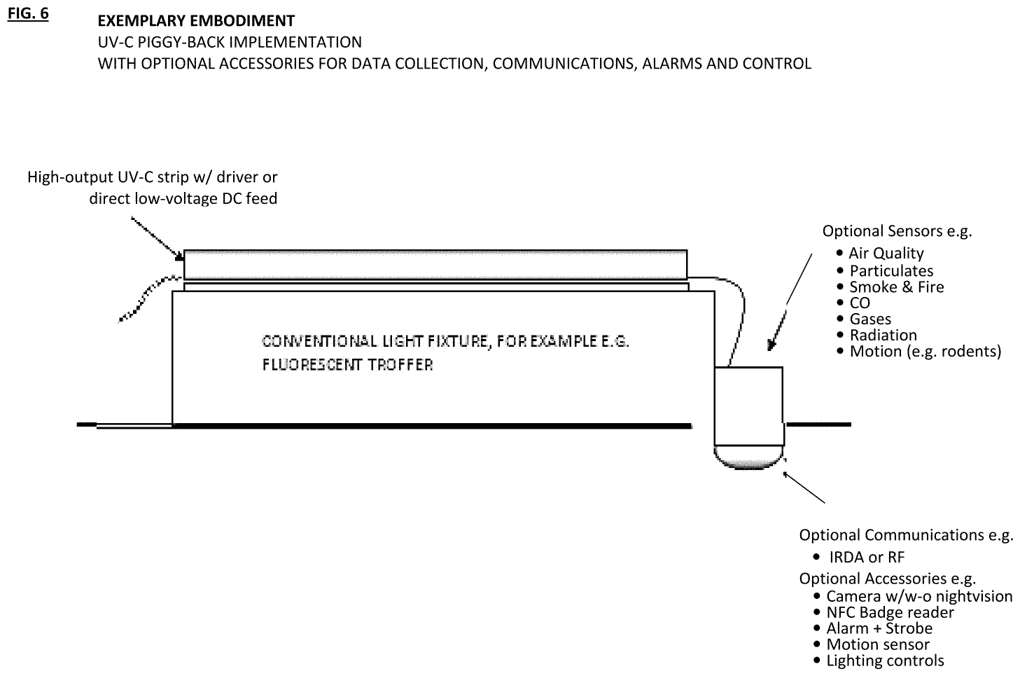

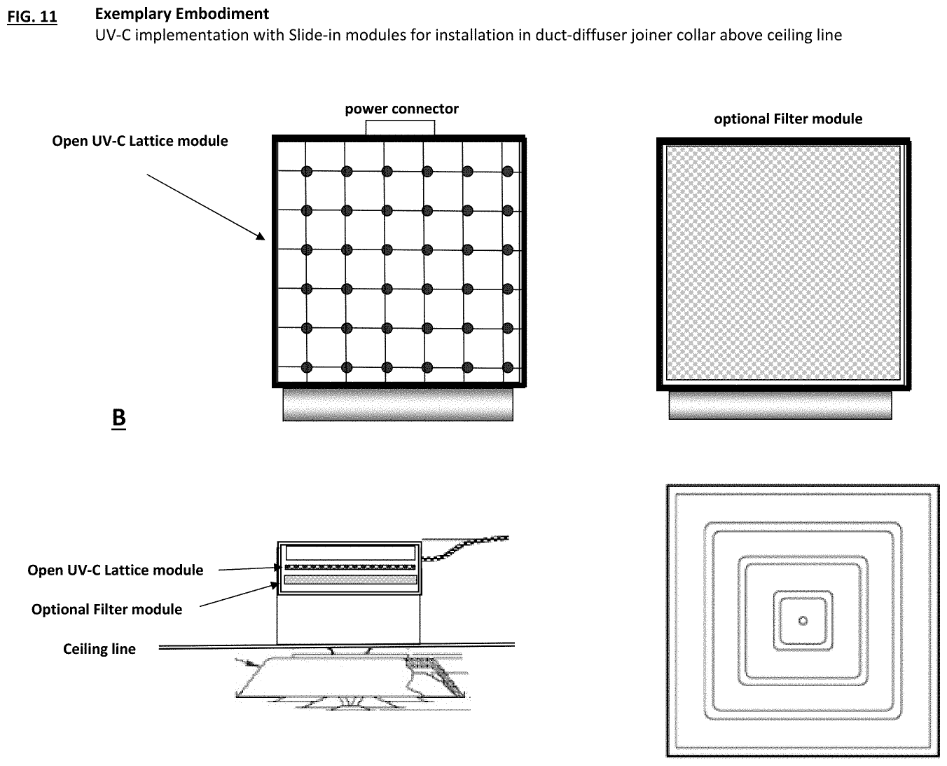

One or more Accessory Devices may be added to at least one exemplary embodiment, as shown, for example e.g., in FIG. 13. At least one embodiment of the invention provides connections for optional plug-in capability to integrate sensors and other accessories into the unit, such as for example e.g. an air quality monitor, camera, transceiver, motorized damper control, as shown for example e.g. in FIG. 6-7. Data gathered by these accessories can be collected, analyzed and transmitted so that the performance of the UV-C apparatus can be assessed and tracked.

While the description and accompanying drawings describe, illustrate and point out novel features as applied to a number of exemplary embodiments of the invention, it may be understood that various omissions, substitutions and changes in the form and details of the apparatuss or algorithms illustrated can be made without departing from the spirit of the disclosure. As may be recognized, certain embodiments or implementations of the inventions described herein may be assembled in a form that provides some, but not all of the features and benefits set forth herein. Some features may be used or practiced separately from others. The scope of exemplary embodiments disclosed herein is represented by the claims in conformity to the specifications and drawings. All changes which come within the meaning and range of equivalency of the claims are to be embraced within their scope. The specifics of packaging of an embodiment in an enclosure do not alter coverage under the claims of the present application so long as a subject device provides equivalent functionality.

Depending on the embodiment, certain acts, events, devices or functions of any of the processes or algorithms associated with the embodiments described herein may be performed in a different sequence, may be added, merged, or left out altogether (e.g., not all described operations or events are necessary for the practice of the invention). Moreover, in certain embodiments, operations or events may be performed concurrently, e.g., through multi-threaded processing, interrupt processing, or multiple processors or processor cores or on other parallel architectures, rather than sequentially.

The elements of embodiments and implementations of the invention described above may be combined, removed, or the layers reordered or rearranged, to create different embodiments of the present application. A combination of such elements or embodiments to create a particular embodiment of the invention would be within the scope and possession of the invention and knowledge of the inventor or those skilled in the art, and is considered to be covered under the scope of the present disclosure.

An embodiment and/or application-specific implementation of an embodiment that includes a set of interoperating active devices and which provides functionality equivalent to that of the invention described herein, irrespective of the manner in which it is housed or the components selected, is assumed to be covered under the claims of the invention.

Numerous modifications and adaptations of the invention disclosed herein will be apparent to those skilled in the art without departing from the spirit and scope of the present application. Further combinations of the different exemplary embodiments and implementations would be within the scope and possession of the invention and knowledge of the inventor or those skilled in the art, and are considered to be covered under the scope of the present disclosure

The figures and drawings included herewith depict some, but not all, of the possible embodiments and application-specific implementations of the invention, and have been selected to illustrate representative combinations of embodiments of the invention. The figures are not intended to depict all possible combinations, embodiments or application-specific implementations of embodiments. None of the drawings is to any scale.

For simplicity of illustration in the figures, details of wiring, connectors, power connections and component mounting are not shown, as these are well-known in the field.

To one skilled in the art, it will be apparent that various omissions, substitutions and changes in the form, details and placement of the devices, or alternative software algorithms, included in the specifications and illustrations can be made without departing from the spirit of the disclosure. As can be recognized, certain embodiments of the inventions described herein can be configured in a form that does not provide all of the features and benefits set forth herein, as some features can be used or practiced separately from others. The scope of certain inventions and embodiments disclosed herein are indicated by the appended claims as a synopsis of the descriptions and drawings. All changes which come within the meaning and range of equivalency of the claims are to be embraced within their scope.

We claim:

1. A method of sterilizing a moving fluid with an apparatus comprising:
a distribution system for a fluid;
at least one UV-C source installed in said distribution system;
a baffle installed in said distribution system in proximity to a UV-C source that directs the fluid in a circuitous path around said UV-C source in order to increase UV-C exposure time of said fluid moving through said distribution system; and
a communicating sensor to monitor at least one parameter of the operation of the apparatus.

2. The sterilization method of claim 1 where the apparatus further incorporates at least one electrochemical cleansing agent activated by UV light.

3. The sterilization method of claim 1 where the apparatus further incorporates at least communicating sensor, including but not limited to for example an air-quality monitor, smoke detector or video camera.

4. The sterilization method of claim 1 where the apparatus further incorporates an electrostatic device that produces ozone to augment the antimicrobial efficacy of said Apparatus.

5. The sterilization method of claim 1 where said fluid contains a medical/biological component, including but not limited to blood, medication or serum.

6. The sterilization method of claim 1 where said fluid is breathable air moving through an HVAC system.

7. The sterilization method of claim 1 where a pipe containing a moving fluid is made of a transparent material that permits exposure to UV-C from a source located outside said pipe.

8. The sterilization method of claim 1 where the invention is incorporated into a section of fluid distribution duct or pipe during fabrication to be incorporated into a new or existing conventional distribution system for said fluid.

9. The sterilization method of claim 1 where the invention is incorporated into a section of said fluid distribution system that controls the egress of said fluid from said distribution system, e.g. for example, a diffuser in a forced-air HVAC distribution system.

10. The sterilization method of claim 1 where the circuitous baffle is transparent and helical in shape.

11. The sterilization method of claim 1 where the apparatus is contained in a housing, the exterior of which contains at least one source of ambient light visible to the human eye.

12. The sterilization method of claim 11 where said ambient light source is able to produce a variety of colors.

13. The sterilization method of claim 12 where said color produced by said ambient light source is remotely-controllable and can be programmed using a microcomputer.

14. The sterilization method of claim 12 where the apparatus further incorporates at least communicating sensor, including but not limited to for example an air-quality monitor, smoke detector or video camera.

15. A method of sterilization to deliver a quantity of UV-C radiation sufficient to disinfect at least one specific site of the surface or the interior of an animate body.

16. The sterilization method of claim 15 where the fiber-optic strand is incorporated into a medical (including dental) instrument.

17. The sterilization method of claim 15 where a fiber-optic strand is used to deliver UV-C to a potential infection site in a medical procedure on the skin or interior of an animate body.

18. A method of sterilization where at least one computer-controlled element, such as a damper, in a fluid distribution system can be manipulated remotely to increase or decrease the flow of said fluid in order to hold said fluid in a chamber to be disinfected by UV-C.

19. The sterilization method of claim 18 where the said fluid is breathable air.

20. The sterilization method of claim 18 where at least one communicating sensor/actuator monitors and adjusts the flow of fluid through the Apparatus.

21. The sterilization method of claim 18 where data from a communicating sensor/actuator is analyzed by a microcomputer to adjust the rate of flow of a fluid in the distribution system.

22. The sterilization method of claim 18 where a microcomputer controlling said flow control element is activated by at least one signal from at least one other communicating sensor/actuator.

23. The sterilization method of claim 22 where one or more computer-controlled flow control elements are operated in coordination with another computer-controlled flow control element in the fluid delivery system to maintain a suitable flow of sanitized fluid delivered to a given location.
